(19) United States
(12) Patent Application Publication
(10) Pub. No.: US 2021/0322539 A1
(43) Pub. Date: Oct. 21, 2021

(54) Cure to new viruses and other diseases that do not have current cure

(71) Applicant: Yigal Adir, Glendale, CA (US)

(72) Inventor: Yigal Adir, Glendale, CA (US)

(21) Appl. No.: 16/873,472

(22) Filed: Apr. 17, 2020

(51) Int. Cl. A61K 39/215; A61K 39/21; C12Q 1/70

(52) U.S. Cl. CPC A61K 39/215; C12Q 1/70; A61K 39/21

(57) ABSTRACT

New approach to enable to find quick and efficient cure to new viruses and other diseases that do not have current cure. To avoid epidemic and save lives of people

### TECHNICAL FIELD

This invention relates to find cure to new viruses and other diseases that do not have current cure to save lives of people

### BACKGROUND

Current medical professions are lacking the ability to find quick cure to new viruses and other diseases like coronavirus and HIV and to prevent epidemic

### SUMMARY

The present invention is directed to find quick and efficient cure to new viruses that do not have current cure and other diseases to save lives and to prevent epidemic. And to prevent government shut down of activities because of the new viruses. It can help to save normal government functions and save billions of dollars of economic losses

### BRIEF DESCRIPTION OF THE DRAWINGS

FIG. 1, Show a cure to viruses by getting antivirus cure from animals that can resists the Viruses

FIG. 2. Show a cure to viruses by getting anti viruses cure by Isolating anti viruses cure from blood of animals that have anti viruses and other defense mechanisem in their Bloods

### INVENTION

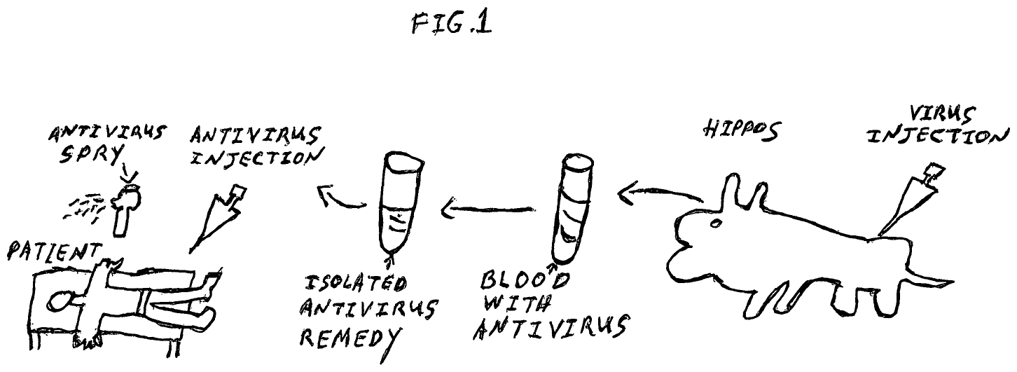

As shown in FIG. 1. Injecting the new viruses like the coronavirus and other viruses to animal that can resist it like Hipposes, Snow Leopards, Amur Leopards, Puma, Dogfish Sharks, Dolphins, horses, buffalos, Ostrich+Ostrich Eggs, Elephants, Bears

We can take the blood from the above animals. And we can Isolate the Anti viruses components from their blood. In case of the ostrich is from their Eggs. And we can use it and we can duplicate it in the medical Lub. And we can Improve its Formula. We can Inject or Spray the Anti Viruses components that we got from the above animals and from the medical Lub to the parts of the patient that have the virus. it will help to heal the patient.

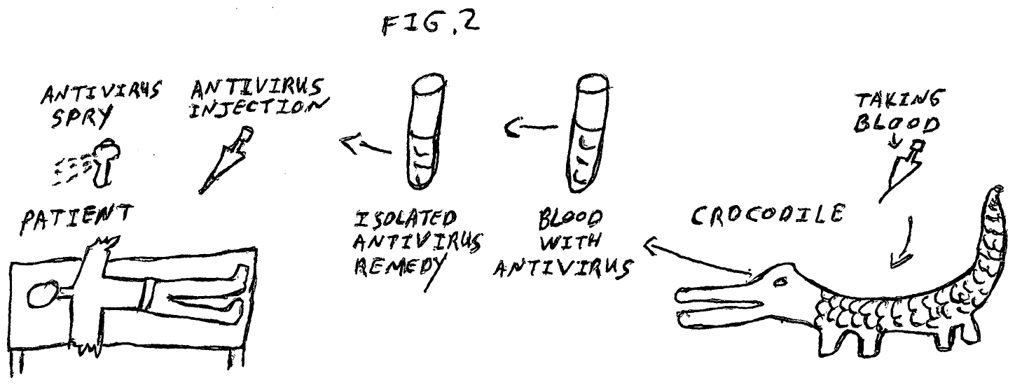

As shown in FIG. 2. We can take the blood from animals that have anti viruses and other defense components in their blood like Crocodiles that have 16 defense components in their bloods against viruses and other deadly diseases like HIV. And blood from Hyena, And blood from Vulture that have defense components against viruses and other deadly diseases in their blood.

We can Isolate the Components that Destroy viruses and we can use it and we can duplicate it. And we can Improve the Formula in the medical Lub.

We can Inject the Isolate Anti Viruses or Spray it on the parts of the patient that have the virus. it will help to Heal the patient

I claim:

1. Cure to virus can be produce by injecting the virus to a live animal that can Resist the virus and produce Anti Virus protection in its blood streams

2. Isolating the Anti Virus protection from the blood of the resistant animal, will provide potential cure against the virus. By using it as it is or duplicating chemical copy, and by improving the formula in medical Lub

3. Injecting or Spraying the Anti Virus components in to the parts of the patient that have the virus will help to Heal the patient

4. Some animals have natural virus resistance in their blood stream. we can isolate the components in the blood stream. we can use it and we can duplicate. chemical copy, and by improving the formula in medical Lub. we can use it to cure the patient and fight the virus

5. We can Inject or Spray the Anti Virus components in the parts of the patient that have the virus it will help to Heal the patient

6. We can study the structure of the virus chemistry and we can develop Anti Agent that can Block the virus ability to Function
